(19) United States
(12) Patent Application Publication
(10) Pub. No.: US 2006/0257432 A1
(43) Pub. Date: Nov. 16, 2006

(54) Pharmaceutical formulations of xanthogenates and inhibitors of viral nucelic acid replication

(76) Inventor: Eberhard Amtmann, Heidelberg (DE)

Correspondence Address: BORDEN LADNER GERVAIS LLP, 1100-100 QUEEN ST, OTTAWA, ON, K1P 1J9, CA

(21) Appl. No.: 10/572,950

(22) PCT Filed: Sep. 9, 2004
(86) PCT No.: PCT/EP04/10044

§ 371 Date: Mar. 21, 2006

(30) Foreign Application Priority Data
Sep. 17, 2003 (DE) 103 43 365.1

(51) Int. Cl. A61K 31/522; A61K 31/44; A61K 31/4745; A61K 31/473; A61K 31/265; A61K 9/00

(52) U.S. Cl. 424/400; 514/298; 514/305; 514/290; 514/512; 514/263.31; 514/263.32

(57) ABSTRACT

The invention relates to pharmaceutical formulations of xanthogenates and agents containing these formulations for the treatment of viral, tumor or autoimmune diseases. The pharmaceutical formulations contain a xanthogenate of formula I 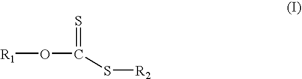
wherein R₁ represents an optionally substituted aryl or alkyl residue and R₂ represents a metal atom, an optionally substituted alkyl, alkoxy, amino or ammonium group or halogen, and an inhibitor of viral nucleic acid replication such as e.g. aciclovir, valaciclovir, penciclovir, famciclovir, and optionally a carrier substance reducing the irritating effect of the xanthogenate and optionally an adjuvant enhancing the activity.

The invention relates to pharmaceutical formulations of xanthogenates in combination with inhibitors of viral nucleic acid replication and agents containing these formulations for the treatment of viral diseases.

Xanthogenates, in particular tricyclodecane-9yl-xanthogenate (D609), are known as substances with antiviral and antitumor activity, e.g. from “DNA and RNA virus species are inhibited by xanthates, a class of antiviral compounds with unique properties” Sauer-G; Amtmann-E; Melber-K; Knapp-A; Muller-K; Hummel-K; Scherm-A, in Proc-Natl-Acad-Sci-U-S-A. 1984 June; 81(11): 3263-7; “Selective killing of tumor cells by xanthates” Amtmann-E; Sauer-G, in Cancer-Lett. 1987 June; 35(3): 237-44, and U.S. Pat. No. 4,602,037.

The pharmaceutical utilization of xanthogenates with antiviral and antitumor activities is impeded by the fact that relatively high agent concentrations are required in order to demonstrate efficacy in an animal model. However, since the concentration of the agent is limited both for pharmacological and technical reasons, even the maximal usable concentrations achieve only a limited healing effect. The same problem applies to common antiviral inhibitors, such as aciclovir, valaciclovir or famciclovir.

We have surprisingly found that a synergistic enhancement of the effect is obtained by combining xanthogenate derivatives, such as D609, and inhibitors of viral nucleic acid replication, such as aciclovir. In the presence of low, antivirally ineffective concentrations of the xanthogenate, the effect of aciclovir in cell culture was observed to be increased up to five-fold. In animal experiments, the combination of D609 and aciclovir achieved survival of all HSV-1-infected animals. Each active ingredient applied separately achieved only partial healing.

The present invention thus solves the aforementioned problem by providing a pharmaceutical formulation that contains a xanthogenate and an inhibitor of viral DNA or RNA replication.

The formulation contains a xanthogenate of general formula I 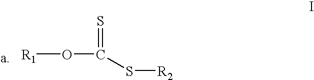
wherein R₁ represents an optionally substituted aryl or alkyl residue.

R₁ preferably represents an adamantyl, norbornyl, tricyclodecyl, benzyl, linear or branched C₃-C₂₀ alkyl, C₃-C₂₀ cycloalkyl, furyl, pyridyl, anthracyl, naphthyl, phenanthryl, perinaphthyl or quinuclidinyl residue, whereby the aforementioned linear or branched C₃-C₂₀ alkyl residue can be substituted by a hydroxyl, a C₁-C₄ alkoxy group, a halogen atom or an amino group, and the aforementioned C₃-C₂₀ cycloalkyl residue can also be substituted by a hydroxyl, a C₁-C₄ alkoxy or a C₁-C₄ alkyl group, a halogen atom or an amino group. Particularly preferred for R₁ are cyclododecyl, dodecyl, undecyl, decyl, tricyclo[5,2,1,0^{2,6}]-decyl, nonyl, octyl, bicyclo[2,2,1]-heptyl, cyclohexyl, hexyl, toluyl residues. Particularly advantageous is an exo/exo-tricyclo[5.2.1.0^{2,6}]-decyl residue.

R₂ represents a metal atom, an optionally substituted alkyl, alkoxy, amino or ammonium group or halogen. Preferably, R₂ represents a mono- or multivalent metal atom, a linear C₁-C₆ alkyl residue, a hydroxy-substituted C₁-C₆ alkyl residue, a C₁-C₆ alkoxy residue, an amino group, a C₁-C₆ alkylamino residue, a di-(C₁-C₆ alkyl)amino residue, a tri-(C₁-C₆ alkyl)ammonium residue, a halogen, 2,3-dihydroxypropyl or hydroxy-(C₁-C₆ alkoxy)methyl. Particularly advantageous are sodium and potassium salts and dimethylglycyl and methyl esters.

Preferably, the inhibitor of viral nucleic acid replication is a nucleoside analogue, and particularly advantageously it is bromodeoxyuridine (BudR), fluorodeoxyuridine (FudR), aciclovir, valaciclovir, penciclovir or famciclovir.

The inhibitor of viral nucleic acid replication can also be an inhibitor of viral helicase.

The inhibitor of viral nucleic acid replication can also be an inhibitor of a cellular enzyme.

Formulations containing 0.1 to 10 parts of inhibitor of viral nucleic acid replication per one part of xanthogenate have proven to be especially useful. Particularly advantageous is a xanthogenate-to-inhibitor of viral nucleic acid replication ratio of 1:1.

The formulation according to the invention preferably contains, in addition, an ionic detergent as an effect-enhancing adjuvant such as is described in U.S. Pat. No. 4,851,435. A fatty acid with 6-19 C atoms or its salt is particularly preferred as adjuvant. Particularly preferred are potassium salts of decanoic, undecanoic or lauric acid. The activity-enhancing adjuvant can also be a sulphate with an aliphatic residue of 8-18 C atoms. Particularly preferred is sodium lauric acid sulphate. Moreover, deoxycholic acid or a pharmaceutically tolerable salt thereof or a phosphonic acid can be used as the adjuvant.

Moreover, it is preferred to incorporate the xanthogenate in lipid- or steroid-based carrier substances in accordance with WO 96/14841. Incorporation into a carrier substance improves the tolerability of the agents. In this context, the carrier substance is particularly a steroid such as cholesterol, cholestanol, cholanic acid, chondrillasterol, and α, β, γ sisterol.

It is particularly preferred for xanthogenate and adjuvant, if any, to be mixed with the carrier substance in accordance with DE 101 17 728. Cholesterol is particularly preferred. Phospholipids, in particular phosphatidylcholine, phosphatidylserine, phosphatidylinositol or stearylamine are also suited for use as carrier substance.

A formulation containing aciclovir, the Na or K salt of decanoic acid, and exo/exo-tricyclo[5,2,1,0^{2,6}]-9yl-xanthogenate is particularly preferred. In particular, this formulation contains one part xanthogenate, one part potassium salt of decanoic acid, and one part aciclovir.

Another particularly preferred formulation contains phosphatidylcholine or cholesterol, the Na or K salt of decanoic acid, exo/exo-tricyclo[5,2,1,0^{2,6}]-9yl-xanthogenate, and aciclovir. In particular, this formulation contains one part xanthogenate, one part decanoic acid, four parts phosphatidylcholine or cholesterol, and one part aciclovir.

According to claims 11 to 16, the present invention also provides agents, containing the pharmaceutical formulation for the treatment of viral, tumor or autoimmune diseases. In addition, these agents contain common carrier substances and/or common excipients. It is also possible that other active ingredients are contained therein provided they interfere neither with the effect nor the stability of the xanthogenates and inhibitors of viral nucleic acid replication.

Particularly preferred are agents in the form of ointments, whereby a lipophilic substance is used as the ointment base. Preferably, vaseline is used as the ointment base.

The pharmaceutical formulation according to the invention and agents containing them are suitable for the treatment of viral, tumor, and autoimmune diseases.

The following examples illustrate the invention in more detail without limiting the invention.

### EXAMPLE 1

Synergistic Enhancement of the Inhibitory Effect of Aciclovir on the Proliferation of Herpes Simplex Virus by the exo/exo Isomer of tricyclo[5,2,1,0^{2,6}]-9yl-xanthogenate.

Human lung carcinoma cells (Calu-6) were infected with 30 plaque-forming units of herpes simplex virus (Type-1, strain ANG) in Linbro plates. Cell culture medium (MEM containing 10% fetal calf serum, 0.85 g/l sodium bicarbonate, and 0.5% carboxymethylcellulose) containing either 0, 5 or 10 μg/ml exo/exo-tricyclo[5,2,1,0^{2,6}]-9yl-xanthogenate (D609) was added two hours after infection. The cultures were treated with aciclovir at the same time. All samples were processed as quadruplicates. The cells were incubated for 48 h at 37° C. under CO₂ gassing (5%). Then the medium was decanted, the cells were fixed with 3% formalin and stained with 0.5% crystal violet. After drying at room temperature, the number of plaques formed was determined.

In the cultures containing no D609 the number of plaques counted was 32.75±11. In the presence of 5 or 10 μg/ml D609 the number of plaques counted was 32±3 plaques and 33±6 plaques, respectively. This means that D609 had no effect on plaque formation at these concentrations. In the presence of aciclovir at a concentration of 0.16 μM the number of plaques was reduced to 30±11. This effect was not statistically significant (student's t-test p=0.4).

In the presence of 10 μg/ml D609 and 0.16 μM aciclovir, the number of plaques was reduced to 9.5±5.1. This effect was statistically significant (student's t-test p=0.014).

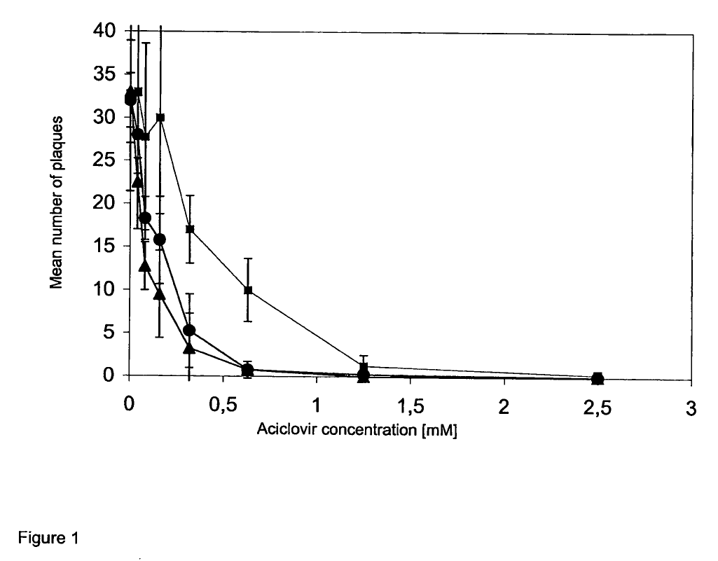

The results are illustrated in FIG. 1, in which the mean number of plaques is plotted over the aciclovir concentration. The series of measurements without D609 is indicated by squares, the series of measurements with 5 g/ml D609 by cycles, and the series of measurements with 10 g/ml D609 by triangles. It is clearly evident that the presence of D609 at concentrations that are ineffective by themselves provides for the onset of the effect of aciclovir at substantially lower concentrations, at which aciclovir alone does not show an effect.

### EXAMPLE 2

Enhancement of the Effect of Aciclovir on the Course of Experimental Infection with Herpes Simplex Virus in Mice by the exo/exo Isomer of tricyclo[5,2,1,0^{2,6}]-9yl-xanthogenate.

One part (by weight) D609, one part (by weight) potassium salt of decanoic acid, and four parts (by weight) cholesterol were mixed in a mortar. Subsequently, propyleneglycol (final concentration 10%) and vaseline were added such that the final concentration of D609 was 5%. The same procedure was used to produce ointments containing 5% aciclovir or 5% D609 and 5% aciclovir. A placebo ointment containing neither of the two agents was produced using the analogous procedure.

Ten mice (Balb-C strain) each were shaved on their upper leg and six scratches in the skin were made with a cannula in an area of 5×5 mm. Subsequently, a cotton bud was used to apply 50 μl of a HSV-1 suspension (Wal strain, 10⁸ plaque-forming units/ml). The treatment (twice daily) was initiated four days after infection. The symptoms, “widespread lesions”, “hind leg paralysis”, and survival were recorded in the protocol.

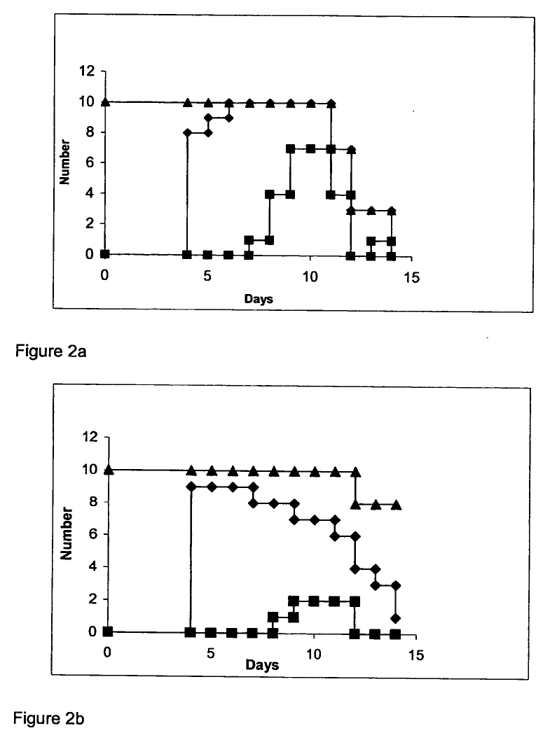

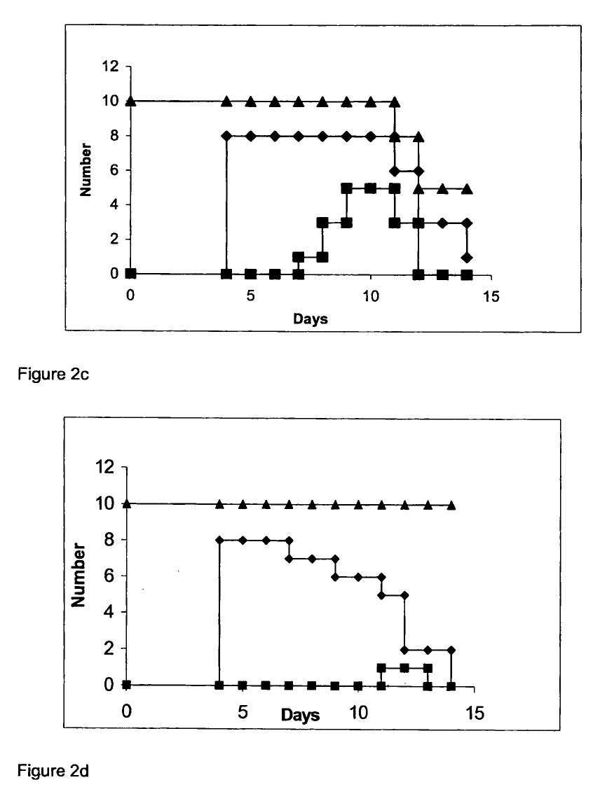

The result is shown graphically in FIGS. 2a to 2d. The plot shows the number of animals displaying the symptoms indicated over the number of days after infection. Triangles indicate the surviving animals in each case, squares the animals with hind leg paralysis, and diamonds the animals with widespread lesions.

FIG. 2a shows for treatment with the placebo ointment containing no agent that only 3 animals survived after 14 days and that all animals had shown widespread lesions and 7 animals had shown paralysis. In FIGS. 2b and 2c, treatment with D609 (2b) or aciclovir (2c) is shown to render the survival rate higher, and lesions and paralysis almost completely healed. For the combination of D609 and aciclovir, FIG. 2d shows that all animals survived and lesions and paralysis were completely healed and manifested to a lesser degree.

Accordingly, the combination preparation is clearly more efficacious than the individual preparations. The combination preparation achieves survival of all animals and a lesser degree of symptom manifestation and clearly more rapid healing of symptoms.

1. Pharmaceutical formulation, comprising
a xanthogenate of formula I 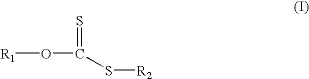
wherein R₁ represents an optionally substituted aryl or alkyl residue, and R₂ represents a metal atom, an optionally substituted alkyl, alkoxy, amino or ammonium group or halogen,
an inhibitor of viral nucleic acid replication,
optionally an adjuvant enhancing the activity of the xanthogenate, and
optionally a carrier substance reducing the irritating effect.

2. Pharmaceutical formulation according to claim 1, wherein R₁ is an adamantyl, norbornyl, tricyclodecyl, benzyl, linear or branched C₃-C₂₀ alkyl, C₃-C₂₀ cycloalkyl, furyl, pyridyl, anthracyl, naphthyl, phenanthryl, perinaphthyl or quinuclidinyl residue, whereby the said linear or branched C₃-C₂₀ alkyl residue can be substituted by a hydroxyl, a C₁-C₄ alkoxy group, a halogen atom or an amino group, and the said C₃-C₂₀ cycloalkyl residue can be substituted by a hydroxyl, a C₁-C₄ alkoxy, a C₁-C₄ alkyl group, a halogen atom or an amino group.

3. Pharmaceutical formulation according to claim 2, wherein R₁ is a cyclododecyl, dodecyl, undecyl, decyl, tricyclo[5,2,1,0^{2,6}]-decyl, nonyl, octyl, bicyclo[2,2,1]-heptyl, cyclohexyl, hexyl or toluyl residue.

4. Pharmaceutical formulation according to claim 1, wherein R₂ is a sodium or potassium atom or a dimethylglycylester or methylester group.

5. Pharmaceutical formulation according to claim 1, wherein the inhibitor of viral nucleic acid replication is a nucleoside analogue.

6. Pharmaceutical formulation according to claim 5, wherein the inhibitor of viral nucleic acid replication is selected from aciclovir, valaciclovir, pencidovir, and famciclovir.

7. Pharmaceutical formulation according to claim 1, comprising 1 to 10 parts inhibitor of viral nucleic acid replication per one part xanthogenate.

8. Pharmaceutical formulation according to claim 1, comprising an ionic detergent as adjuvant.

9. Pharmaceutical formulation according to claim 1, comprising deoxycholic acid or a pharmaceutically tolerable salt thereof as adjuvant.

10. Pharmaceutical formulation according to claim 1, comprising a phosphonic acid as adjuvant.

11. Pharmaceutical formulation according to claim 1, comprising cholesterol as carrier substance.

12. (canceled)

13. Pharmaceutical formulation according to claim 1, wherein the xanthogenate is tricyclo[5,2,1,0^{2,6}]-decane-9-yl-xanthogenate, the carrier substance is cholesterol or phosphatidylcholine, the adjuvant is the sodium or potassium salt of decanoic acid, and the inhibitor of viral nucleic acid replication is selected from aciclovir, valaciclovir, penciclovir, and famciclovir.

14. Pharmaceutical formulation according to claim 13, wherein the inhibitor of viral nucleic acid replication is aciclovir.

15. Pharmaceutical formulation according to claim 1, comprising one part xanthogenate, one part inhibitor of viral nucleic acid replication, four parts carrier substance, and one part adjuvant.

16. Pharmaceutical formulation according to claim 1, further comprising a lipophilic substance as excipient for administration as an ointment.

17. Pharmaceutical formulation according to claim 16, wherein the lipophilic substance is vasoline.

18. Pharmaceutical formulation according to claim 7, comprising 2 to 4 parts inhibitor of viral nucleic add replication per one part xanthogenate.

19. Pharmaceutical formulation according to claim 8, wherein the ionic detergent is a fatty add with 6 to 19 C atoms or an alkylsulphate with 8 to 18 C atoms.

20. Method for the treatment of viral, tumor, or autoimmune diseases comprising administering to a patient in need thereof an effective amount of a xanthogenate of formula I 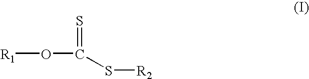
wherein R₁ represents an optionally substituted aryl or alkyl residue, and R₂ represents a metal atom, an optionally substituted alkyl, alkoxy, amino or ammonium group or halogen, and an effective amount of an inhibitor of viral nucleic acid replication.

21. Method according to claim 20, wherein R₁ is a cyclododecyl, dodecyl, undecyl, decyl, tricyclo[5,2,1,0^{2,6}]-decyl, nonyl, octyl, bicyclo[2,2,1]-heptyl, cyclohexyl, hexyl or toluyl residue and R₂ is a sodium or potassium atom or a dimethyl-glycylester or methylester group.

22. Method according to claim 20, wherein the inhibitor of viral nucleic acid replication is selected from aciclovir, valaciclovir, penciclovir, and famciclovir.

23. Method according to claim 20, further comprising cholesterol or phosphatidylcholine as a carrier substance, and sodium or potassium salt of decanoic acid as an adjuvant, and wherein the xanthogenate is tricyclo[5,2,1,0^{2,6}]-decane-9-yl-xanthogenate, and the inhibitor of viral nucleic acid replication is selected from aciclovir, valaciclovir, penciclovir, and famciclovir.
